(19) United States
(12) Patent Application Publication
(10) Pub. No.: US 2009/0136603 A1
(43) Pub. Date: May 28, 2009

(54) ACTIVE COMPONENT FRACTION OF HIGH ANTIVIRAL ACTIVITY

(76) Inventors: Kathrin Kabrodt, Bernburg (DE); Ingo Schellenberg, Dessau (DE)

Correspondence Address: LAW OFFICES OF KARL HORMANN, 86 SPARKS STREET, CAMBRIDGE, MA, 02138, US

(21) Appl. No.: 12/225,942

(22) PCT Filed: Mar. 30, 2007
(86) PCT No.: PCT/DE07/00580

§ 371 Date: Oct. 2, 2008

(30) Foreign Application Priority Data
Apr. 3, 2006 (DE) 10 2006 015 573.4

(51) Int. Cl. A61K 36/708; A61P 31/12

(52) U.S. Cl. 424/773

(57) ABSTRACT

A method of producing active component fractions from dried and ground root material of a species of the rheum family by extraction with ethanol, removal of lipophilic minor components and methanol, followed by shaking in ethyl acetate and separating the combined ethyl acetate phase into a limited number of fractions by columnar chromatography in the presence of an eluant.

### BACKGROUND OF THE INVENTION

1. Field of the Invention

The invention relates to active component fractions of high antiviral activity for virus inactivation and disinfection.

2. The Prior Art

The antiviral action of condensed tanning agents has not only been described but it has also been therapeutically used. Esquenazi, D., Wigg, M. D. et al. (2002), Research in Microbiology 153: 647-652, researched the antiviral activity of condensed tanning agents extracted from dried fibers of a ripe coconut (Cocos nucifera L.) on infected HEp-2 (laryngeal cancer cell line and VERO cell cultures. They were able to demonstrate that polyphenols derived from catechins impede the activity of acyclovir-resistant herpes simplex virus species 1 (HSV-1-ACVr). As well as a complete extract, several chromatographically pre-separated fractions were applied. The complete extract and a fraction with monomeric to trimeric procyanidines displayed the strongest antiviral activity which the authors claimed to be the result of a disturbed adsorption of the viruses at the cell line. Cheng, H. Y., Lin, C. C. et al. (2002), Antivir Chem Chemother. 13(4): 223-229 examined the antiviral properties of prodelphinidin B-2-3′-0-gallate, isolated from green tea, on herpes simplex species 2 cultivated from VERO cells. Aside from the disturbance of the adsorption to the host cell resulting from the active agent, the significant reduction of the infection is also traced to an impaired penetration of the virus. The same results were obtained by Cheng, H. Y., Lin, T. C., et al. on the same virus-model with the digalloylated prodelphinidin compound B-2-3,3′-0-gallate which was isolated from myrica rubra, (2003) Planta Med. 69(10): 953-956. Various monomeric flavan-3-oles and, more particularly, EGCG, isolated from green tea displayed antiviral activity against an adenovirus, as demonstrated by Weber et al., (2003) Antiviral Res. 58(2): 167-173. The highest efficiency was attained by applying EGCG at the transition from the early to the late phase of the viral infection.

The antiviral activity in connection with flavenoids and proanthocyanidins isolated from Crataegus sinaica was especially pronounced in the range of the dimeric to tetrameric PC. In this context, a complete HSV-1 titer reduction was observed. The antiviral activity was determined by way of the inhibition of cytopathic effects on VERO cells infected by herpes simplex virus, as described by Shahat, A. A. et al., (2002). Planta Med. 68: 539-541.

The antiviral activity of extracts of Geranium sanguineum L. enriched by polyphenols (tannins, flavonoids, catechins and proanthocyanidins against an influenza virus was examined by Sokmen, M., et al., (2005) Life Sciences 76: 2981-2993. They used a complete methanolic extract and ethyl acetate and butanol fractions derived therefrom to affect, in vitro, the reproduction of human influenza viruses (A/Aichi virus in MDCK=Madin-Barby canine kidney cells). The reduction of the cytopathic effects was measured. The complete extract as well as the BuOH fractions displayed a significant reduction of the virus-induced cyclopathic effects. An ethanolic extract of the subterraneous parts of Rh. officinale displayed, in vitro, antiviral activity against HSV. The minimum inhibiting dose was about 100 μg/ml, described by Wang, Z. W., et al., (1996), Zhongguo Zhong Yao Za Zhi 21(6): 364-366.

Even though a number of well characterized antiviral substances and compounds exists, there is yet a demand for new active substances. On the one hand, the known active substances either display an insufficient activity or they are not well tolerated and, on the other hand, more and more virus strain are becoming resistant. The known extract compounds derived from various rheum species display an insufficient antiviral effect.

### OBJECT OF THE INVENTION

It is an object of the invention to provide active substance fractions from various species of the rheum family with an increased active compound potential for producing antiviral formulations.

The invention aims to provide active component fractions of high antiviral activity which are characterized by being substantially without side-effects.

### BRIEF SUMMARY OF THE INVENTION

In accordance with the invention, the object is accomplished by active compound fractions of high antiviral activity derived from various rheum species and which are characterized by the active substance fractions being derived by extraction of dried and ground root samples with methanol, the resultant aqueous phase being cleared of lipophilic minor components by complete removal of the methanol, the purified aqueous phase being extracted with ethyl acetate and the united ethyl acetate phase being separated into a limited number of active component fractions by columnar chromatography and the active component fractions of like spectral content being combined.

It was extraordinarily surprising to find that individual rhubarb fractions derived by chromatographic separation from the complete ethyl acetate extract of rheum spec. affect a significant virus inactivation. In biology, viruses are known as genetic elements which are structured as nucleic acids which are replicated as foreign components in cells of living beings (host cells) independently of the nucleic acids thereof by the replication mechanisms of these cells. Virus nucleic acids are either deoxyribonucleic acids (DNA) or ribonucleic acids (RNA). Viruses may occur as nucleic acid within a host cell or, as free particles, outside of cells, the latter form being called virion.

Virions consist of a nucleic acid molecule surrounded by a coating (capsid). In the case of some viruses, the virion, in addition to its protein coating, is provided with additional components, e.g. a lipo protein coating.

The viruses or corresponding model viruses used for examining the antiviral activity of polyphenolic fractions made from rhubarb will be presented hereafter:

### Parvoviruses

Porcine parvovirus (PPV) was used as a model virus for the human pathogenic parvovirus B19. The porcine parvovirus is a member of the parvoviridae and measures about 18-25 nm. The structure of the virus capsides is icosahedral.

The virus particles consist of 60 capsomers and are provided, at their corners, with protein protrusions measuring about 7 nm in length. The virions are not covered by a membrane. The single-strain linear viral DNA genome is contained in the interior of the capsides.

### Flaviviruses

The hepatitis C virus is assigned to the flavivirus group. Since it requires an extraordinary effort to reproduce the HCV in cell cultures, virus inactivation studies have to resort to model viruses. Its genome structure suggests that HCV belongs to the family of flaviviruses and may, therefore, like the virus of bovine diarrhoea (BVDV), be assigned to the genus pestivirus. The infectious viruses of bovine diarrhoea have a diameter of from 40-50 nm. The spherical capsides are surrounded by a coating membrane. The genome consists of single-strain RNA.

### Togaviruses

The Semliki Forest virus (SFV) which is a member of the togavirus family, was also used as a model virus for HCV. The infectious particles have a diameter of from 60-80 nm and consist of icosahedral or spherical capsides which are surrounded by a membranous coating. The genome of togaviruses consists of single-strain RNA.

### Herpes Viruses

Latent infections may be caused by human pathogenic herpes viruses. Following an infection, herpes viruses may persist for a lifetime in lycocytes. The pseudo rabies virus (PRV) was used as the model virus for herpes viruses in the context of these examinations.

The virions of herpes viruses are of a diameter of from 150-200 nm and consist of a total of more than 30 protein structures. The genome in virions is a linear double-strained DNA.

The antiviral effect of tanning agents may be traced to their interaction with protein structures on the surface of the viruses or of the host which impedes the virus adsorption or penetration on or into the host cell and, therefore, the replication of viruses. The virus surface may, on the one hand, be a coating of pure protein. On the other hand, complex viruses are surrounded by a lipid-containing coating which is interspersed by glycoproteins (spikes). These glycoproteins contribute to the infectious activity of the virus. They are synthesized by glucosidases. Tanning agents, on the one hand, act antivirally because of their reaction with proteins in the membrane, and, on the other hand, their toxicity is traced to the inhibition of the glucosidases and, as a result thereof, to a change of the intact surface structure of the virus necessary for an infection. This would also explain why coated viruses, such as herpes simplex, react with greater sensitivity to tanning agents than do viruses without a coating.

### DESCRIPTION OF THE SEVERAL DRAWINGS

The novel features which are deemed to be characteristic of the invention are set forth with particularity in the appended claims. The invention itself, however, in respect of its structure, construction, lay-out and design, as well as manufacturing techniques and technology, together with other objects and advantages thereof will be best understood from the following description of the preferred embodiments when read with reference to the appended drawings, in which:

FIG. 1 depicts an extraction flow diagram of the preparation of dried ground rhubarb roots;

FIG. 2 depicts the chromatographic separation of the ethyl acetate fraction 4 sc-produced from rheum palmatum MAXIM (fraction 4, variant 11). In each case, the upper trace shows the absorption at 280 nm whereas the lower trace shows it at 242 nm;

FIG. 3 depicts the chromatographic separation of ethyl acetate fraction 5 sc-produced from rheum spec. rural species Poland 1976 (fraction 5, variant 25) absorption at 280 nm;

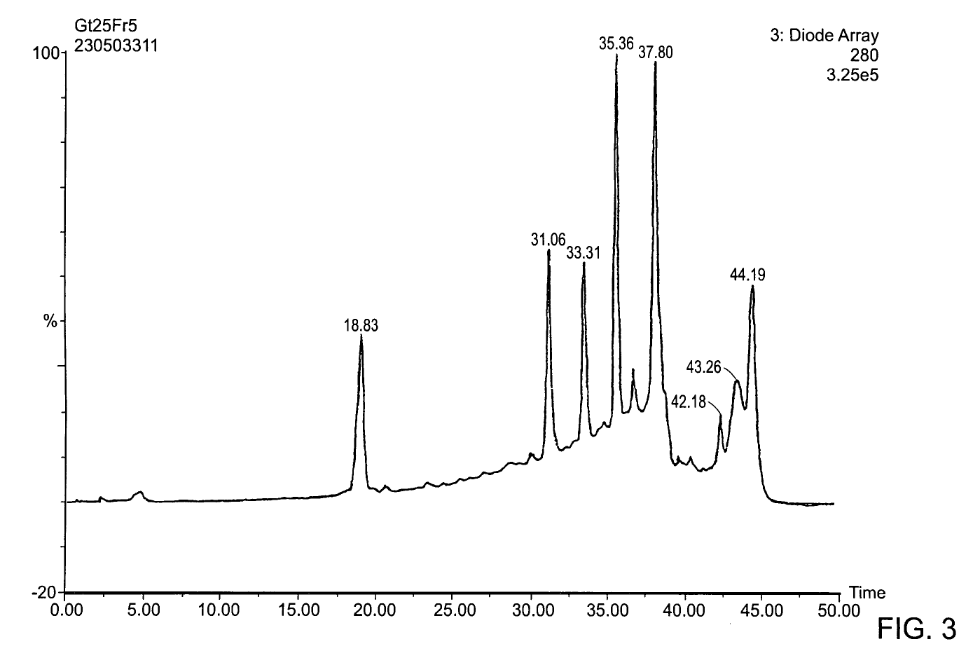

FIG. 4 depicts the chromatographic separation of ethyl acetate fraction 9 sc-produced derived from rheum spec. rural species Poland 1976 (fraction 9, variant 25), absorption at 280 nm;

FIG. 5 shows the inhibition of the viral activity of SFV, PPV, BVDV and RPV by rheum faction 4, variant 11;

FIG. 6 depicts the inhibition of the viral activity of SFV, PPV, BVDV and PRV by rheum fraction 5, variant 25; and

FIG. 7 depicts the inhibition of the viral activity of SFV, PPV, BVDV and PRV by rheum fraction 9, variant 25.

### DETAILED DESCRIPTION OF THE INVENTION

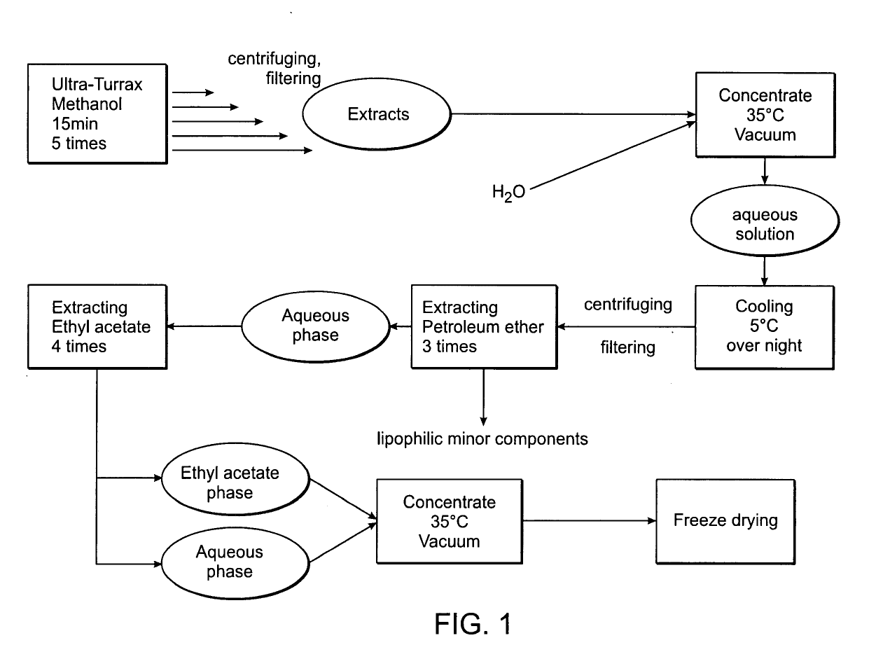

FIG. 1 depicts the extraction flow diagram of the preparation of dried and ground rhubarb roots. For producing the active components, recourse is had to a modification of a known extraction protocol as described, for instance, in German patent specification DE 101 32 936 A1.

To produce the aqueous initial phase, methanol is used as the primary extraction agent instead of acetone/water, and the methanolic extracts are used thereafter. Following a multi-stage extractive preparation, a preparatory columnar chromatographic separation of the complete ethyl acetate extract was carried out.

1 kg of a dried and ground root sample was subjected to a quintuple centrifugal extraction in an Ultra-Turrax with about 8,000 ml of methanol being used for 15 min each for the extraction of the rhubarb root material. The collected extracts were then concentrated in a rotary evaporator, in vacuum, at 35° C., and after adding 500 ml of water, the methanol was removed completely. For precipitating disturbing lipophilic substances the aqueous extract was maintained in nitrogen at 5° C. in a refrigerator over night. On the following day the precipitates were removed by centrifuging. Thereafter, further lipophilic minor components were removed by a three-fold extraction of the aqueous solution with 500 ml of petroleum ether each. The aqueous phase thus purified was then extracted four time with 750 ml of ethyl acetate following which increased low oligomerized compounds were to be present in the combined ethyl acetate phases as were, in the remaining aqueous phase, compounds of a higher degree of oligomerization. The ethyl acetate as well as the aqueous phase were then concentrated at 35° C. under vacuum in a rotary evaporator, placed in a little water followed by freeze-drying.

For further use, the freeze dried extracts enriched by tanning agent were stored at −21° C. in a nitrogen atmosphere.

The columnar chromatographic (sc) preparatory separation of the ethyl acetate phase and of the aqueous phase proved to be indispensable for reducing the overall spectrum of polyphenolic compounds to a limited number of substances.

For the preparatory isolation of proanthocyanides, separation with a Sephadex LH-20 has been practiced for a considerable time. Ethanol or alcohol/water mixtures are established as eluants. Acetone/water mixtures (7:3; v/v) are useful for separating polymeric substances.

The ethyl acetate phase was dissolved in methanol i.e. methanol/water and applied to the column (length 100 cm, diameter 5 cm) and successively eluated with ethanol, methanol and acetone/water (7:3; v/v). A MPLC unit with a sample feeding pump was used for this purpose. Following a DC check of the produced test tube fractions, the fractions of ethyl acetate phase of the same spectral content were combined for the individual genomes.

Following sc separation of the ethyl acetate phase, eleven fractions resulted in the case of GT11 (Rheum palmatum M.) and 14 fractions in the case of GT25 (rheum spec. country species Poland).

For purposes of applications in accordance with the invention fraction 4, variant 11 (rheum palmatum M.), fraction 5 and 9, variant 25 (rheum spec. country species Poland) were used.

The following conditions are true for characterizing the fractions obtained by columnar chromatography and are considered to be most effective in respect as regards their action:

Chromatography

| Stationary phase: | Synergy 4 m Polar RP 80, 250 × 2.0 mm; | |
|---|---|---|
| Mobile phase: | A 1% formic acid; | |
| | B methanol; | |
| Flow: | .3 ml/min. | |
| Gradient conditions: | Time (min) | % B |
| | 0 | 0 |
| | 10 | 10 |
| | 40 | 50 |
| | 41 | 0 |
| | 55 | 0 |

| | |
|---|---|
| Detection: | DAD |

It was possible by calibration with corresponding standard substances to quantify the following substances in the sc-pre-separated fractions from the ethyl acetate phase—to the extent it was present:

epigallo catechin
catechin
procyanidine B1
epicatechin
procyanidine B2
epigallocatechin gallate
epicatechin gallate

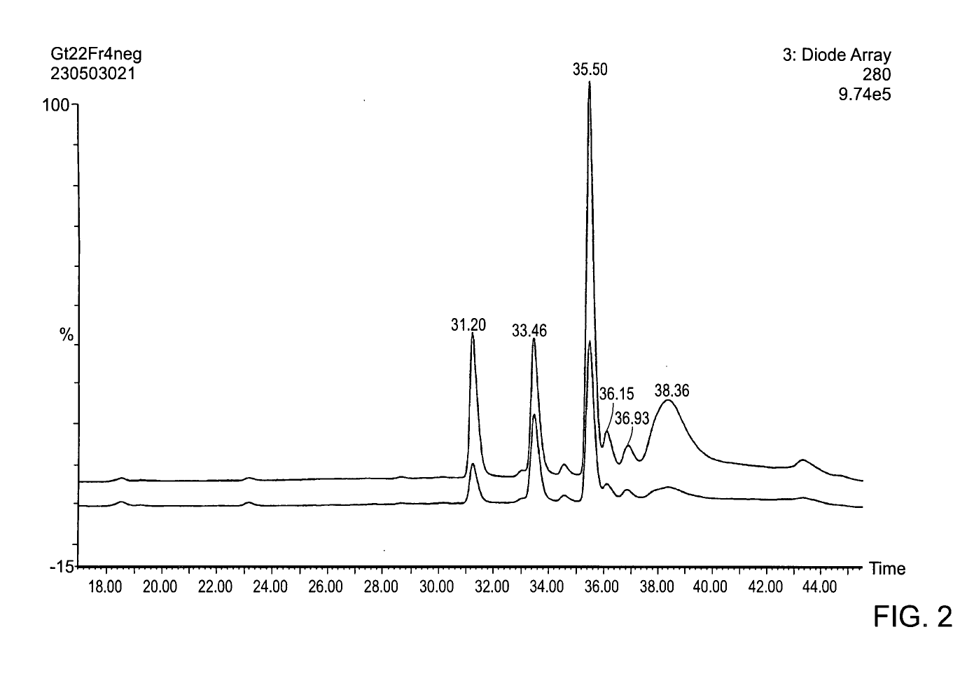

FIG. 2 depicts the chromatogram of the most effective fractions from the sc separation of the ethyl acetate phase of rheum palmatum MAXIM. The upper trace shows the absorption at 280 nm, the lower trace at 242 nm. Both wavelengths were used for the quantification, since the two procyanidines displayed significantly higher absorption intensities at 242 nm.

In rheum palmatum MAXIM it was possible quantitatively to define:

catechin
procyanidine B1
procyanidine B2
epicatechin gallate

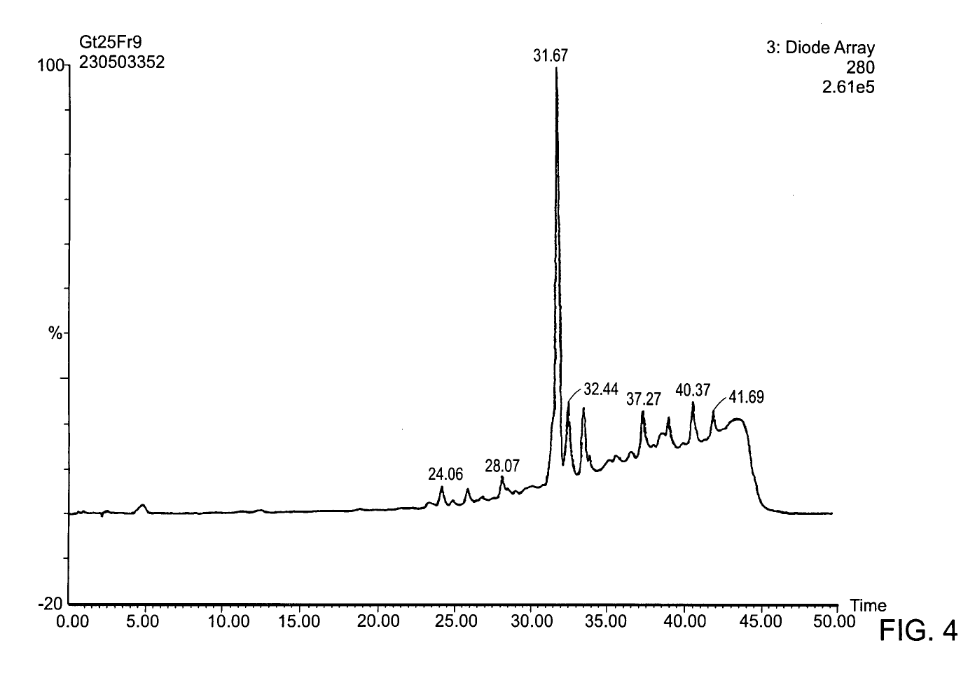

These data were confirmed by the simultaneous recording of the total ion current by means of mass spectrometry. Moreover, the identification of the substances was carried out by comparison of the mass spectra of the standard substances with the mass spectra of the peaks detected during the standard retention times. Compared with all other fractions, fraction 4 isolated from rheum palmatum MAXIM displayed the highest content of quantifiable substances both as to sum and, particularly, to the highest content of epicatechin gallate. FIGS. 4 and 4 display chromatograms of the most effective fractions from the sc separation of the ethyl acetate phase of rheum spec. country species Poland 1976 (fractions 5 and 9, variant 25). It was not possible to specify this species with greater precision. The chromatograms depict the UV-trace at 280 nm.

In rheum spec. country-species Poland 1976 is was possible in fraction 5 quantitatively to define:

procyanadine B1
procyanadine B2
epicatechin gallate

The substances available as standards were no longer available in fraction 9. Higher oligomeric polyphenolic compounds such as digalloylized dimeric procyanidines and trimeric procyanidines of different degrees of gallyolation were detected by mass spectometry, in addition to further compounds described for rhubarb, such as derivatives of toluoylene (stilbene).

The rheum species is a plant which can be found anywhere on earth and which has adapted in different species to its environment.

The following is a list of all species of rhubarb suitable for accomplishing the object of the invention.

Variant 01: Rh. officinale BAILL.; Dolomites (Muenster)
variant 02: Rh. officinale BAILL.; Varutot (Hungary)
variant 03: Rh. officinale BAILL.; Kirovsk (Russia)
variant 04: Rh. officinale BAILL.; medicinal rhubarb, Wisley (England)
variant 05: Rh. officinale BAILL.; Dolomites (Muenster)
variant 09: Rh. palmatum L.; Petersburg (Russia)
variant 10: Rh. rhabarbarum L.; The Sutton
variant 11: Rh. Palmatum l.f.florc rubro MAXIM, Petersburg (Russia)
variant 12: Rh. rhabarbarum L.; Holstein Blut
variant 13: Rh. rhabarbarum L.; Utrecht (Holland)
variant 14: Rh. rhabarbarum L.; Uppsala (Sweden)
variant 15: Rh. rhabarbarum L.; KVDR 1986
variant 16: Rh. rhaponticum L.; Kirovsk (Russia)
variant 17: Rh. rhaponticum L.; Dnepropetrovsk (Russia)
variant 18: Rh. rhaponticum L.; Kirovsk (Russia)
variant 19: Rh. rhaponticum L.; Uppsala (Sweden)
variant 20: Rh. rhaponticum L.; Petrograd (Russia)
variant 21: Rh. rhaponticum L.; Taplozek (Finland)
variant 22: Rh. rhabarbarum L.; Kirovsk (Russia)
variant 23: Rh. rhabarbarum L.; China 1956
variant 24: Rh. rhabarbarum L.; KVDR 1987
variant 25: Rh. spec. rural species Poland 1976
variant 26: Rh. alexandra VEITCH.; Kirovsk (Russia)
variant 27: Rh. wittrockii LUNDSTR.; Petrogra (Russia)
variant 29: Rh. emod. WOLL.; Petrograd (Russia)
variant 30: Rh. leucorum; Wroclaw, (Poland)
variant 31: Rh. leucorrhizum PALL.; Bukarest (Rumania)
variant 32: Rh. maximowiczii LOSINSK.; Petrograd (Russia)
variant 33: Rh. tataricum l.f.lasi (Rumania)
variant 34: Rh. undulatum L.; Pruhonice (Czech Republic)
variant 35: Rh. altaicum LOSINSK.; Petrograd (Russia)
variant 36: Rh. altaicum LOSINSK.; Moscow (Russia)
variant 37: Rh. old rural species (Germany)
variant 38: Rh. old rural species (Germany)
variant 39: Rh. old rural species (Poland)
variant 40: Bastard Rh. Rhab. xRh. raponticum
variant 41: Rh. alexandra VEITCH.; Petrograd (Russia)
variant 42: Rh. officinale BAILL.; rural species (Slovakian Tatra Mountains).

The use of the active component fractions in accordance with the invention will be explained in greater detail in the following embodiments:

For executing the experiments the following materials and methods were applied for inactivating the viruses.

### Viruses and Cells

Model viruses for human pathogenic virus: PPV, BVDV, SFV, PRV

The viruses listed in the following tables were used for the inactivation experiments. Also listed are the cell cultures in which the viruses were reproduced and titrated.

TABLE 1
List of the Viruses Used in the Experiments, their
Properties as well as the Cell Lines Corresponding
to their Reproduction and Titration

| | | Virus Model | | Size | Cell |
|---|---|---|---|---|---|
| Virus | Family | for | Genome | [nm] | Line |
| PPV | Parvoviridae | B19 | ssDNA | 18-26 | PK13 |
| Porcine | | | | | |
| Parvovirus | | | | | |
| BVDV | Flaviviridae | Hepatitis C | ssRNA | 40-50 | MDBK |
| Bovine Viral | | | | | |
| Diarrhea | | | | | |
| Virus | | | | | |
| SFV | Togaviridae | Hepatitis C | ssRNA | 60-80 | Vero |
| Semliki | | | | | |
| Forest | | | | | |
| Virus | | | | | |
| PRV | Herpesviridae | Herpes | dsDNA | 150-200 | ML |
| Pseudo | | Viruses | | | |
| rabies | | (human) | | | |
| Virus | | | | | |

The following partial steps were carried out for the virus inactivation:

### Cultivation of the Cells

The used cell lines (indicator cells, see Table 2) are adhering, i.e. cells growing in mono layers. The cells are subject to contact inhibition and stop growing as soon as they are confluent, i.e. as soon as a dense cell bed has grown. For this reason the cells were subcultivated when the culture had reach a confluence of 70%. Cultivation of the cells was carried out in an incubator at 37° C., 5% carbon dioxide and 95% relative humidity. All work was performed under sterile conditions. Prior to use, the media were provided with the appropriate additives (see Table 2). All additives were stored at −20° C. In order to destroy any present complement factors, the fetal calf serum (FKS) prior to its use was additionally inactivated by placement in an aqueous bath for 30 min at 56° C.

TABLE 2
Cell Lines for the Reproduction of Viruses

| Cell Line | Medium | Medium Additives | Cultivation in |
|---|---|---|---|
| MDBK | DMEM | .4% (v/v) NSP | 75 cm² Cell |
| Vero | | 1% (v/v) L-Glutamine | Culture Vials |
| ML | | 5% (v/v) FKS | |
| PKB | DMEM | .4% (v/v) NSP | 75 cm² Cell |
| | | 1% (v/v) L-Glutamine | Culture Vials |
| | | 10% (v/v) FKS | |

### WST-Assay for Defining the Cell Toxicity

The metabolic activity of living and growable cells can be defined by means of the cell proliferation reagent WST-1. The test is based upon the intracellular reduction of tetrazolium salt in the dark red dye Formazan contained in the WSR reagent by mitochondrial dehydrogenases. The quantity of dye may thus be directly correlated to the number of metabolistically active cells. A cytostatic effect can be demonstrated by a reduced cell proliferation. The absorption was defined with the Wallace Victor at a wavelength of 450 nm. In each cavity of each of a micro titer plate (96 cavities) 8,000 cells of cell line PK13, MDBK, Vero and ML were disseminated. The last gap (gap 12) contained medium only and served as a zero sample. In addition, a positive control (medium and cell only) and two further controls in which the medium contained 5% and 10%, respectively, of ethanol were run. This was necessary since for better solubility the polyphenolic fractions required a share of at least 5% ethanol in the solvent.

Three rhubarb fractions were tested: Fraction 4 isolated from rheum variant 11, fraction 5 and fraction 9 isolated from rheum variant 25 in the levels of concentration 0.0025%, 0.005%, 0.1% and 0.5%. 50 μl. of each level of concentration of each fraction was pipetted for purposes of a double determination to the different cells (except the controls) which were incubated for 24 hours. Thereafter, 20 μl. of WST-1 reagent was added by pipetting to each cavity and the absorption was measured immediately. Thereafter, for 240 minutes the absorption was measured every 30 minutes.

### Determination of the Virus Titer of PPV, BVDV, SFV and PRV.

The determination of the virus titer was carried out by end point titration. For this purpose, the virus-containing material was thinned until no cytopathic effect (CPE) was any longer recognizable. The determination of the virus titer is segregated into the following four operational steps.

### 1. Dissemination of the Cells

Determination of the virus titer was carried out by infection of the corresponding cells (Table 3) with different dilutions of the given virus sample. A microtiter plate was required for each sample taken. A day prior to the treatment of the given virus with the polyphenols the cells were disseminated into the microtiter plates. For a micro titer plate 15 ml of cell suspension were required at a dissemination of 150 l. per cavity. The plates were stored in an incubator (37° C., 5% CO₂, 95% relative humidity).

### 2. Production of the Series of Dilutions for the Titration

Serial 1:5 dilutions in DMEM (1% glutamin, 0.4% NSP, no FKS) were produced from the virus samples to be tested. In a test tube rack of a 96 tube capacity (8×12 tubes) 600 I. of DMEM were placed into the tubes (except those in the front row). Row 1 contained 1 ml of the virus sample to be tested or 500 I. virus plus 500 I. of the concentration level of the polyphenolic fractions. Commencing from row 1, 150 I. were pipetted into the second row by a multichannel pipette, were well mixed three times, and 150 I. from it were pipetted into row 3, etc. The tips were changed at each dilution step. The 12^{th} row contained medium only and was used as a negative control.

### 3. Titration

Titration took place in the microtiter plates which had been seed the day before. 50 l. of the individual dilution stages from the 96 test tube rack were applied to each of the microtiter plates. For each dilution eight copies of it were tested. Depending upon the incubation period of the treated virus, the plates were incubated for 2 to 5 days. Thereafter a microscopic test for cytopathic effects (SPE) was performed.

### 4. Determination of the Tissue Culture Infective Dose; TCID50/ml

Following their incubation period the plates were examined by microscope for cytopathic effects (CPE) caused by virus infections. Positive reactions in the individual cavities of the microtiter plates were recorded in the protocol.

The calculation was carried out computer-assisted on the basis of the probit analysis. Spearman's and Kärber's formula, simplified, may be used for determining the virus titer:

log₁₀TCID₅₀/volume=X₀−d/2+d/n*ΣX_{I}
X₀₌=logarithm of the smallest dilution at which the test object react positively
d=the dose (dilution) spacing in log₁₀
n=the number of test objects used per dilution
X_{I}=the sum of all test objects, beginning at and including X₀, reacting positively to the infection

The virus titer was calculated for a volume of 1 ml. Since for the end point determination a volume of 50 l. (=½0 ml) was used the result had to be added with log₁₀20. The fact that the viruses were diluted 1:1 by the addition of the polyphenols needed also to be taken into consideration in the calculation.

### Results of the Virus Inactivation

The quantities of infectious viral particles present in the initial suspensions were measured by the conventional titration method as described under 3. In this connection, only the highest dilution of that virus suspension was determined which causes a cytopathologic effect (CPE) in the culture of the indicator cells (50% tissue culture infective doses per ml (log TCID₅₀/ml)). The following images depict the log TCID₅₀/ml values of the virus suspensions, of the controls (positive control: virus in medium with 5% ethanol) as well as of the viruses treated with the different concentration stages of the rheum fractions. Only those concentration stages are shown at which no cytotoxicity was observed with respect to the indicator cells VERO, PK 13, MDBK and ML (these results have not been listed here).

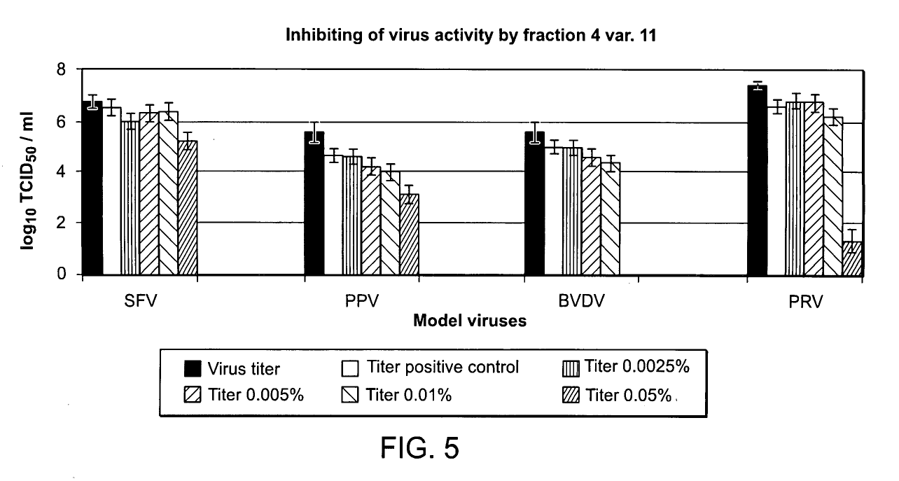

As shown in FIG. 5, at a concentration of 0.05% all model viruses are being significantly inhibited by the fraction 4, variant 11. In view of the fact that even at 0.01% a significant inhibition relative to the pure virus suspension can be observed, the PRV and PPV viruses seem to be the more sensitive ones.

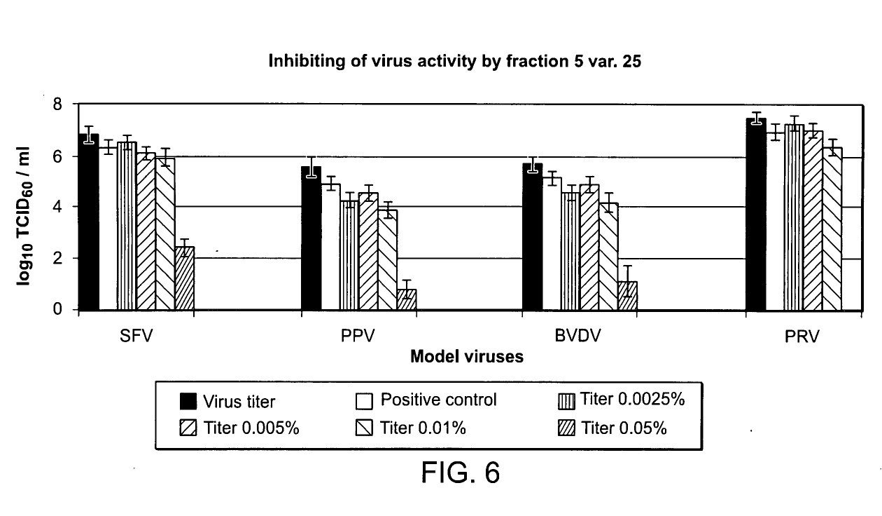

Fraction 5, variant 25 has the strongest inhibiting effect on PPV and BVDV. All shown concentrations from 0.05% to 0.005% (FIG. 6) display a virus-inhibiting effect compared to the virus titer. As regards PRV, only the 0.01% concentration acts in a virus inhibiting manner.

The antiviral effectiveness of fraction 5, variant 25 can be observed on the virus model SFV in the range of concentration between 0.05% and 0.005%.

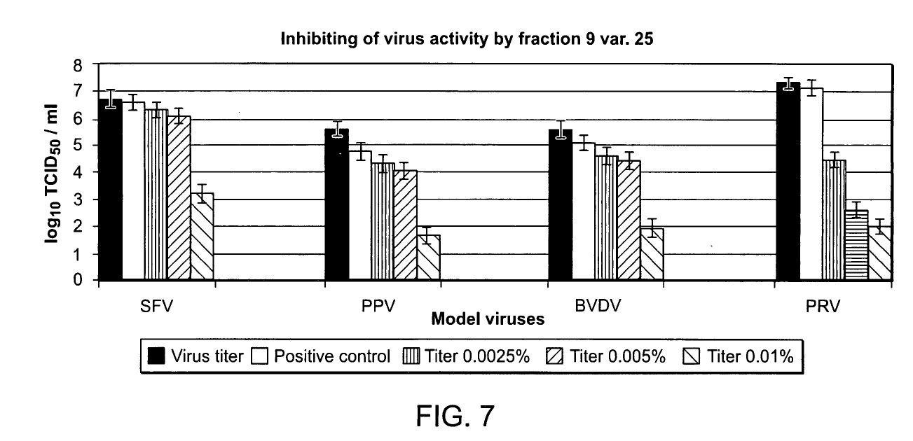

Fraction 9, variant 25 is generally cytotoxically effective beginning at a concentration of 0.05%. The strongest antiviral effects of this fraction can be seen at PRV. As may be seen in FIG. 7, viral activity is reduced by about 40 at the lowest used concentration. At the highest non-cytotoxical concentration a reduction of about 70% of the initial activity is attained.

At PPV and BVDV an inhibition of the viral activity occurs at concentrations between 0.01% and 0.0025%. At the highest used concentrations the viral activity is lowered by about 65% to 70%. In the case of SFV only the 0.01% concentration has a significant virus titer reducing effect.

What is claimed is:

1-5. (canceled)

6. A method of producing active component fractions of high antiviral activity from rheum family species for the inactivation of viruses and disinfection, comprising the steps of:
a) extracting dried and ground root sample of a species of the rheum family with methanol;
b) removing lipophilic minor components from the resultant aqueous phase to obtain a purified aqueous phase;
c) shaking the purified aqueous phase with ethyl acetate;
d) separating combined ethyl acetate phases by columnar chromatography into a limited number of active component fractions;
e) combining the active component fractions of identical spectral content; and
f) separating individual active component fractions for use following a demonstration of the effectiveness of the active component fractions.

7. The method of claim 1, wherein the lipophilic minor component is removed by precipitation at 5° C. under exposure to nitrogen.

8. The method of claim 1, wherein step b) is further includes the step of shaking the aqueous phase with petroleum ether.

9. The method of claim 1, wherein step c) is followed by the further step of concentrating the combined ethyl acetate phases in vacuum.

10. The method of claim 9, wherein the step of concentration is followed by placing the ethyl acetate phases in water and freeze-drying.

11. The method of claim 1, wherein the columnar chromatography is performed with at least one of ethanol, methanol and a mixture of acetone and water as an eluant.
